(19) United States
(12) Patent Application Publication
(10) Pub. No.: US 2013/0131696 A1
(43) Pub. Date: May 23, 2013

(54) TOOL FOR REMOVING STRATUM CORNEUM

(71) Applicant: Takuya Ishida, Las Vegas, NV (US)

(72) Inventor: Takuya Ishida, Las Vegas, NV (US)

(73) Assignee: KOWASHOJI Co., LTD., Shibuya-ku, Tokyo (JP)

(21) Appl. No.: 13/811,209

(22) PCT Filed: Jul. 22, 2010
(86) PCT No.: PCT/JP2010/062305

§ 371 Date: Jan. 18, 2013

(51) Int. Cl. A61B 19/00

(52) U.S. Cl. CPC A61B 19/00
USPC 606/131

(57) ABSTRACT

A tool for easily removing stratum corneum by acting on the stratum corneum to be removed from various directions and in a complicated manner with a complex uneven pattern. The tool is also capable of controlling the amount of stratum corneum to be removed. The tool for removing stratum corneum is formed with the stratum corneum removal face on one side of the plate-like body thereof. Several different groups of grooves are formed on a flat one side of the plate-like body thereof, after which holes are formed by punching from the other side of the plate-like body towards the one side such that the stratum corneum removal face is formed with uneven sections on the periphery of the holes.

### BACKGROUND

1. Technical Field

The present invention relates to a tool for removing stratum corneum or strata cornea (which may also be referred hereinafter to as the stratum corneum removing tool or simply the tool) that is used in removing any stratum corneum that may occur on the sole, heel, elbow or the like of the human body.

2. Description of the Prior Art

There are various types of the tool for removing stratum corneum or strata cornea that have been proposed until the present time.

As one example of the tool for removing stratum corneum, Patent Document 1 that is referred to hereinafter in the “Prior Patent Documents” proposes to provide a tool for removing stratum corneum that includes a round flat plate-like body having holes formed on the substantially spherical projecting surface thereof and a slip or slide preventive means provided on the substantially spherical outer peripheral edge. According to the invention disclosed in Patent Document 1, it is described that the tool is capable of only removing any unwanted stratum corneum from the skin; it will not damage the outer skin of the human body by scratching it roughly; it will not cause the stratum corneum that has been removed to be clogged in the joints on the skin; it can be washed easily and dried in a short time; and it provides the excellent advantages from the aspects of hygiene, safety and usability.

As another example, Patent Document 2 that is referred to hereinafter in the “Prior Patent Documents” proposes to provide a tool for removing stratum corneum that includes a metallic flat plate having a plurality of round holes formed thereon. In this tool, the plurality of round holes may be made by the punching operation. After the punching operation is completed, the projected portion on the rear side of the metallic flat plate may be flattened by the polishing operation so that the resulting depressions of several μm to several ten μm can be left between every two adjoining round holes. The tool can be manufactured in the simple way, and is capable of only removing any unwanted stratum corneum from the human skin effectively.

### PRIOR PATENT DOCUMENTS

Patent Application No. H11-225905

Patent Application No. 2001-204649

### SUMMARY OF THE INVENTION

FIG. 1 is provided to describe the conventional tool for removing stratum corneum 21 that includes a metallic plate-like body 22 having holes formed thereon by the punching operation.

The plate-like body 22 has a flat surface on one side 23 thereof on which a stratum corneum removing face is to be formed, and includes a plurality of holes 26a, 26b, 26c, 26d, etc that are formed by the punching operation so that the holes 26a, etc can be passed through the plate-like body 22 from the other side (rear side) toward the direction shown by an arrow 20. In this way, the stratum corneum removing face is formed on the one side 23 of the plate-like body 22.

By forming the holes 26a, etc by the punching operation, uneven sections are provided around every two adjoining holes 26a, 26b, 26c, 26d, etc formed on the one side on which the stratum corneum removing face is formed. Those uneven sections will be helpful in removing the stratum corneum.

It should be noted, however, that the uneven sections that will be formed around the peripheral edges of every two adjoining holes 26a, 26b, 26c, 26d, etc have virtually the same size because those holes have been formed on the flat side 23 by the punching operation. In addition, those uneven sections can be formed so that they may have virtually the same pattern.

When the tool 21 is used for removing the stratum corneum and if the tool 21 is then only to be moved in a particular direction in order to remove any particular stratum corneum, therefore, one problem is that although the particular stratum corneum may be removed by the uneven sections while the tool 21 is being moved in the particular direction, yet other strata cornea cannot be removed. Another problem is that it is difficult to control the amount of the stratum corneum to be removed because the uneven sections that are provided around the peripheral edges of every two adjoining holes 26a, 26, 26c, 26d, etc have substantially the same size or pattern.

It is accordingly an object of the present invention to provide a tool for removing stratum corneum that is capable of easily removing any particular stratum corneum by acting on the particular stratum corneum to be removed from the various directions even when the tool is only to be moved in a particular direction, and also by utilizing the uneven pattern formed on the stratum corneum removal face in order to act on the particular stratum corneum to be removed from the various directions and yet in the complicated manner by, and is also capable of controlling the amount of stratum corneum to be removed.

The invention according to Claim 1 provides a tool for removing stratum corneum that includes a plate-like body having a stratum corneum removing face formed on one side thereof, wherein
the stratum corneum removing face having;
   several different groups of grooves that are first formed on one flat side of the plate-like body;
   holes that are then formed by the punching operation so that they can be passed through the plate-like body from the other or rear side of the plate-like body toward the one side thereof; and
   uneven sections formed around the peripheral edges of the holes formed on the one side of the plate-like body by the punching operation so that they can correspond to the several different groups of grooves formed on the plate-like body.

The invention according to Claim 2 provides the tool for removing stratum corneum as defined in Claim 1, wherein the several different groups of grooves include:
a first group of grooves extending in the first predetermined oblique direction with every two adjoining grooves being in parallel with each other;
a second group of grooves extending in the second direction across the first direction in which the first group of grooves extend with every two adjoining grooves being in parallel with each other; and
a third group of grooves extending in the third direction across the first direction in which the first group of grooves extend as well as across the second direction in which the second group of grooves extend with every two adjoining grooves being in parallel with each other.

The invention according to Claim 3 provides the tool for removing stratum corneum as defined in Claim 1 or Claim 2, wherein
the holes include a plurality of holes to be formed by the punching operation.

In accordance with the advantages of the present invention, the stratum corneum to be removed can be removed easily by acting on the stratum corneum to be removed from various directions as well as by utilizing the complex uneven pattern formed on the stratum corneum removing face in order to act on the stratum corneum to be removed from the various directions and yet in the complicated manner even when the tool is only to be moved in a particular direction. The amount of stratum corneum to be removed can also be controlled.

### BRIEF DESCRIPTION OF DRAWINGS

FIG. 1 is a diagram for illustrating the conventional tool for removing stratum corneum, in which (a) is a perspective view that represents the state in which the tool is placed before the stratum corneum removing face is to be formed, (b) is a perspective view that represents the state in which the tool is placed after the stratum corneum removing face has been formed, and (c) is an enlarged plan view that represents the state in which the tool is placed with the holes being formed on the stratum corneum removing face.

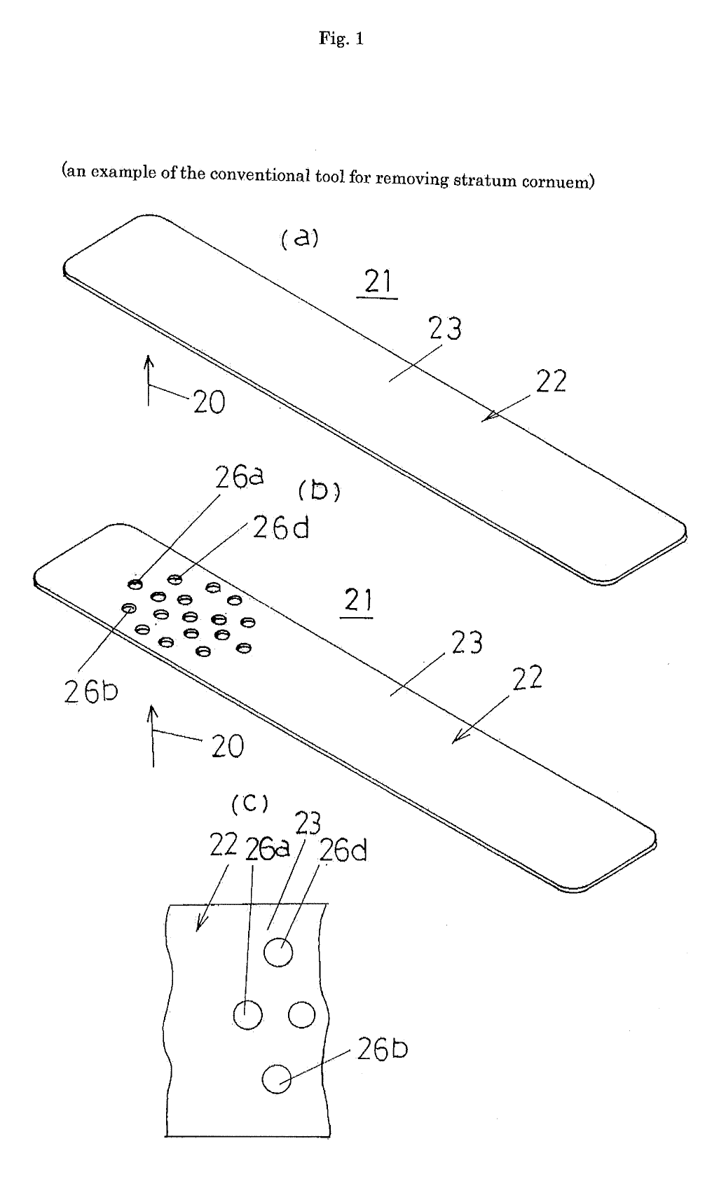

FIG. 2 is a diagram for illustrating the inventive tool for removing stratum corneum, in which (a) is a perspective view that represents one side of the plate-like body on which the stratum corneum removing face is formed and (b) is a perspective view that represents the other side of the plate-like body.

FIG. 3 is a diagram for illustrating the steps of the process during which the inventive tool for removing stratum corneum is being constructed, in which (a) is a plan view showing one side of the plate-like body, (b) is a plan view showing the state in which the third group of grooves extending in the third direction has been formed, (c) is a plan view showing the state in which the first group of grooves extending in the first direction has been formed, (d) is a plan view showing the state in which the second group of grooves extending in the second direction has been formed, and (e) is a plan view showing the state in which a plurality of holes has been formed by the punching operation so that they can be passed through the plate-like body from the other or rear side of the plate-like body toward the one side of the plate-like body on which the stratum corneum removing face is to be formed.

FIG. 4 (a) is an enlarged sectional view for the inventive tool for removing stratum corneum which is taken along the line A-A in FIG. 3 (a), schematically showing the state in which the several different groups of grooves are formed on the side on which the stratum corneum removing face is to be formed although some parts are omitted, (b) is an enlarged perspective view for the inventive tool for removing stratum corneum, schematically showing the hole formed on the stratum corneum removing face, and (c) is an enlarged plan view for the inventive tool for removing stratum corneum, schematically showing how the peripheral edge of the hole is to be formed on the stratum corneum removing face.

### BEST MODE OF EMBODYING THE INVENTION

Now, one preferred embodiment of the present invention will be described by reference to the accompanying drawings.

The tool for removing stratum corneum 1 according to the present invention (which may also be referred to hereinafter as the tool 1 for the sake of simplicity) includes a plate-like body 2 that may be made of any metallic materials and has a stratum corneum removing face 5 that is formed on one side 3 thereof.

The stratum corneum removing face 5 includes several different groups of grooves that are formed on one flat side 3 of the plate-like body 2, and further includes a plurality of holes 6a, 6b, 6c, 6d, etc that are formed by the punching operation so that they can be passed through the plate-like body 2 from the other or rear side 4 of the plate-like body 2 toward the one side 3 thereof. In the specification and accompany drawings, those holes 6a to 6d may be referred to collectively as the “hole 6” hereinafter.

As described above, the several different groups of grooves are formed by the punching operation on the one flat side 3 of the plate-like body 2, and the plurality of holes 6 are further formed by the punching operation so that they can be passed through the plate-like body 2 from the other side 4 of the plate-like body 2 toward the one side 3 thereof. In this way, the uneven sections are formed around the peripheral edges of the holes 6 formed by the punching operation on the one side 3 of the plate-like body 2 on which the stratum corneum removing face 5 is to be formed, and are provided so that they can correspond to those several different groups of grooves which are formed on the one side 3 of the plate-like body 2.

Those uneven sections formed around the peripheral edges of the holes 6 may have many different sizes as well as many different uneven patterns that may depend on the several different groups of grooves. This is because the holes 6 are formed by the punching operation so that they can be passed through the plate-like body 2 from the other side 4 toward the one side 3 on which the several different groups of grooves are formed.

Accordingly, the tool 1 of the present invention can be operated to act against any particular stratum corneum to be removed from the various directions even if the tool 1 is only to be moved in the particular direction. The tool 1 of the present invention can also easily remove any particular stratum corneum by utilizing the complex uneven patterns formed on the stratum corneum removing face 5 in order to act against the particular stratum corneum from the various directions and in the complicated manner. The uneven sections formed around the peripheral edges of the holes 6 and having the many different sizes and many different patterns can also be used to control the amount of the stratum corneum to be removed.

In the tool 1 of the present invention, it is preferred that several different groups of grooves each extending in a different direction should be provided.

For example, the several different groups of grooves may include a first group of grooves 8 extending in the first direction with every two adjoining grooves being in parallel with each other, a second group of grooves 9 extending in the second direction with every two adjoining grooves being in parallel with each other, and a third group of grooves 7 extending in the third direction with every two adjoining grooves being in parallel with each other.

More specifically, it is preferred that the first group of grooves 8, the second group of grooves 9 and the third group of grooves 7 may be provided so that each group of grooves can extend in a different direction.

For example, it is preferred that the direction in which the first group of grooves 8 extend with every two adjoining grooves being in parallel with each other may intersect the direction in which the second group of grooves 9 extend with every two adjoining grooves being in parallel with each other and that the direction in which the third group of grooves 7 extend with every two adjoining grooves being in parallel with each other may intersect the direction in which the first group of grooves 8 extend as well as the direction in which the second group of grooves 9 extend.

The uneven sections formed around the peripheral edges of the holes and having the extremely complicated patterns and complicated sizes can thus be provided by the different groups of grooves 7, 8, 9 formed to extend in the different directions as combined with the holes 6 formed by the punching operation so that they can be passed through the plate-like body 2 from the other side 4 toward the one side 3 of the plate-like body 2 on which those different groups of grooves are formed.

As a result, it will be possible to remove the stratum corneum much more effectively.

It may be appreciated from the above description that for each different group of grooves, the size of every groove, the width of the spacing between every two adjoining grooves, the depth of every groove and the like can be adjusted in various ways, depending on the different expected usages of the tool for removing stratum corneum when it is to be constructed. The form and size of the uneven section that is formed around the peripheral edge of each of the holes 6 may be modified in various ways by changing the size, width, depth and the like as mentioned above. The amount of the stratum corneum to be removed may also be controlled accordingly.

It is thus desirable from the aspect of the stratum corneum removing efficiency that the holes 6 should include a plurality of holes that are formed by the punching operation.

The following describes one preferred embodiment of the present invention by referring to the accompany drawings. It should be understood, however, that the present invention is not limited to the preferred embodiment described herein but various modifications, changes or alternations may be made in various ways without departing from the spirit and scope of the invention as defined in the appended claims.

### Embodiment

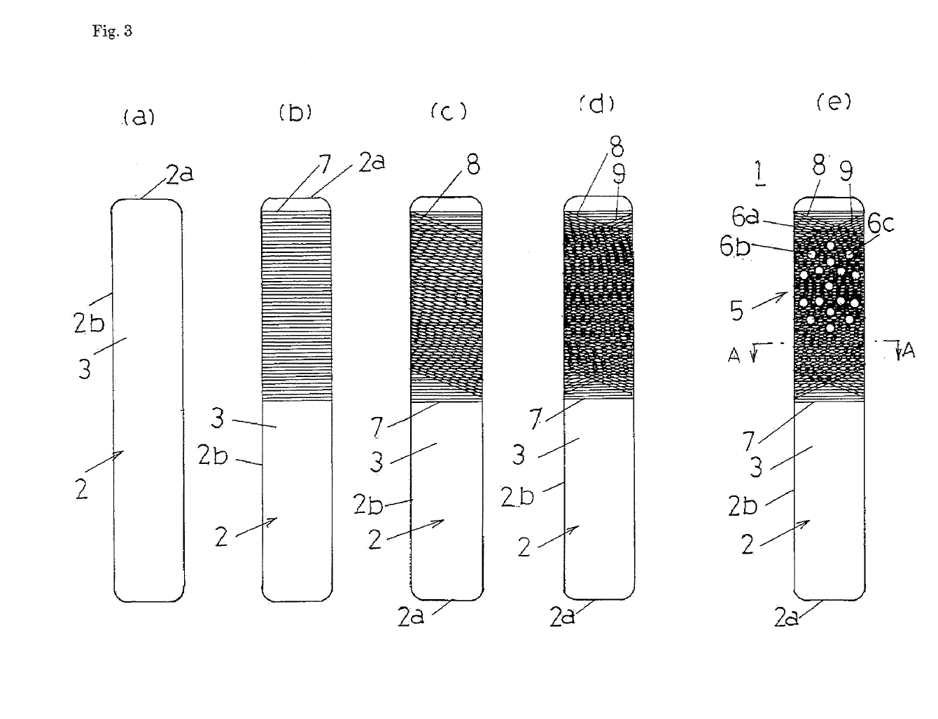

A plate-like body 2 that may be made of metal is provided (FIG. 3 (a)).

The plate-like body 2 has one flat side shown by reference numeral 3 on which a stratum corneum removing face 5 is to be formed, and includes a third group of grooves 7 that will be formed on the one flat side 3 so as to extend in parallel with the shorter edge 2a of the plate-like body 2 (FIG. 3 (b)). The third group of grooves 7 include a plurality of grooves with every two adjoining grooves 7 being in parallel with each other as shown by FIG. 3 (b)). The size of each individual groove 7, the width of the spacing between every two adjoining grooves 7, and the depth of each individual groove 7 can be adjusted in various ways, depending on the different expected usages of the tool 1 when it is to be constructed.

Then, the first group of grooves 8 will be formed on the one side 3 of the plate-like body 2 so that they can extend in the first direction across the direction of the third group of grooves 7 also formed on the one side 3 of the plate-like body 2 (FIG. 3 (c)). In the embodiment shown and described herein, the first group of grooves 8 extend in the oblique direction from the left upper side toward the right side with every two adjoining grooves 8 being in parallel with each other (FIG. 3 (c)). In this case, the size of each individual groove 8, the width of the spacing between every two adjoining grooves 8, and the depth of each individual groove 8 may also be adjusted in various ways, depending on the different usages of the tool 1 when it is to be constructed.

Then, the second group of grooves 9 extending in the second direction will be formed on the one side 3 of the plate-like body 2 so that they can intersect the third group of grooves 7 as well as the first group of grooves 8 (FIG. 3 (d)). In the embodiment shown and described herein, the second group of grooves 9 extend in the oblique direction from the right upper side toward the left side in FIG. 3 (d) with every two adjoining grooves 9 being in parallel with each other. In this case, the size of each individual groove 9, the width of the spacing between every two adjoining grooves 9 and the depth of each individual groove 9 may also be adjusted in various ways, depending on the different expected usages of the tool 1 when it is to be constructed.

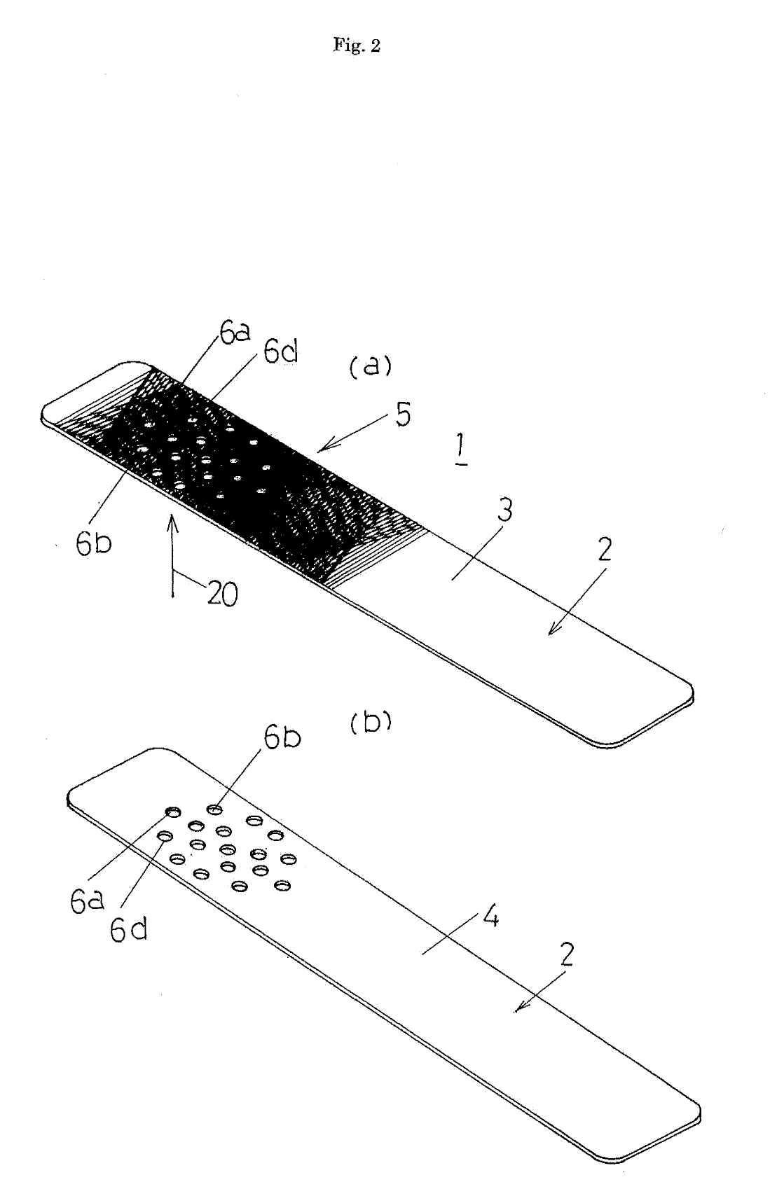

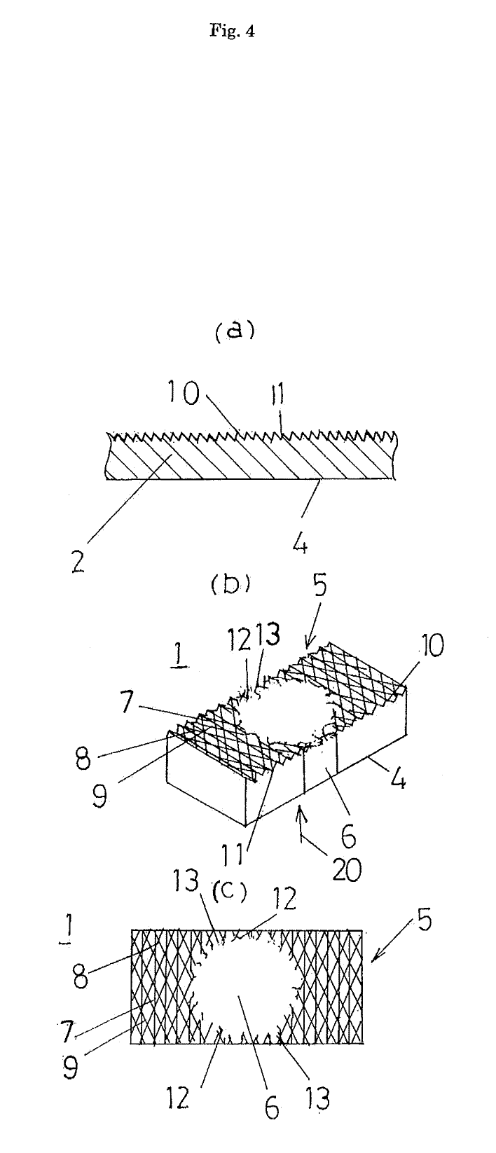

As a next following step, as shown by reference numeral 20 in FIG. 2 (a) and FIG. 4 (b), the holes 6 will be formed by the punching operation so that they can be passed through the plate-like body 2 from the other side 4 of the plate-like body 2 toward the one side 3 on which the stratum corneum removing face 5 is to be formed. As one typical example, this punching operation can be used to form the holes 6. In the embodiment shown and described herein, this punching operation has been used to provide the plurality of holes 6 (FIG. 3 (e)).

As described before, the three different groups of grooves have been formed on the one flat side 3 of the plate-like body 2 so that they can extend in the different directions, respectively (FIG. 3 (a)). Although those different groups of grooves are simplified and are only shown schematically in FIG. 4 (a), the complicated projections 10 and depressions 11 that are represented schematically as being formed on the one side 3 of the plate-like body 2 can be formed as the result of those three different groups of grooves.

Then, the holes 6 will be formed by the punching operation in order to pass through the plate-like body 2 from the rear side thereof. The projections 12 and depressions 13 that are formed around the peripheral edges of the holes 6a, 6b, 6c, 6d, etc correspond to the complicated projections 10 and depressions 11 that have the many different sizes, forms and patterns.

The result is the complicated uneven pattern formed on the stratum corneum removing face 5. Then, the tool 1 of the present invention can be operated to act against any particular stratum corneum to be removed even if the tool 1 is only to be moved in any particular direction. In addition, the tool 1 can easily remove any particular stratum corneum by acting against it from the various directions and in the complicated manner because there is the complicated uneven pattern formed on the stratum corneum removing face 5. Furthermore, the projections 12 and depressions 13, each of which is formed around the peripheral edge of each of the holes 6a, 6b, 6c, 6d, etc, may have the many different sizes, forms and patterns that allow the tool 1 to be used to control the amount of the stratum corneum that can be removed by the tool 1.

In the present invention, the holes have been formed by the punching operation so that they can be passed through the plate-like body from the rear side of the plate-like body toward the one side on which the several different groups of grooves extending in the respective different direction have been provided, and the uneven sections having the complicated forms, sizes and patterns that correspond to the complicated uneven patterns provided by those several different groups of grooves have been formed.

In the embodiment shown and described in FIG. 3, therefore, the three different groups of grooves extending in the respective different directions as described before, that is, the first group of grooves 8, the second group of grooves 9 and the third group of grooves 7 have been formed. It may be appreciated, however, that the present invention is not limited to those different directions. For example, the third group of grooves 7 may be provided so that they can extend in parallel with the longer side 2b of the plate-like body 2.

In the embodiment that has been described so far, the plate-like body 2 is made of metal, but it may be appreciated that the plate-like body 2 may be made of any other non-metal material that has the properties required for the tool for removing stratum corneum.

### DESCRIPTION OF REFERENCE NUMERALS

1 Tool for removing stratum corneum

2 Plate-like body

3 One side of plate-like body

4 Other side of plate-like body

5 Stratum corneum removing face

6 (6a, 6b, 6c, 6c, etc) holes

7 Third group of grooves

8 First group of grooves

9 Second group of grooves

10, 12 Projections

11, 13 Depressions

1. A tool for removing stratum corneum, the tool comprising:
a plate-like body having a stratum corneum removing face formed on one side of the plate-like body, wherein said stratum corneum removing face has:
several different groups of grooves that are first formed on the one side of said plate-like body;
holes that are then formed by a punching operation so that the holes can be passed through said plate-like body from another side of said plate-like body toward said one side; and
uneven sections formed around peripheral edges of said holes formed on said one side of said plate-like body by the punching operation so that the uneven sections correspond to said several different groups of grooves formed on said plate-like body;
wherein said several different groups of grooves include:
a first group of grooves extending in a first predetermined oblique direction with every two adjacent grooves being parallel with each other;
a second group of grooves extending in a second direction across the first direction in which the first group of grooves extends with every two adjacent grooves being parallel with each other; and
a third group of grooves extending in a third direction across the first direction in which the first group of grooves extends as well as across the second direction in which the second group of grooves extends with every two adjacent grooves being parallel with each other.

2. (canceled)

3. The tool for removing stratum corneum as defined in claim 1, wherein the holes include a plurality of holes to be formed by the punching operation.
